(19) United States
(12) Patent Application Publication
(10) Pub. No.: US 2013/0310842 A1
(43) Pub. Date: Nov. 21, 2013

(54) INSTALLATION TOOL FOR BONE SCREW

(71) Applicants: Tobias WINKLER, Binswangen (DE); Marcus Richter, Wiesbaden (DE)

(72) Inventors: Tobias WINKLER, Binswangen (DE); Marcus Richter, Wiesbaden (DE)

(21) Appl. No.: 13/741,432

(22) Filed: Jan. 15, 2013

(30) Foreign Application Priority Data
May 15, 2012 (DE) 102012104207.1

(51) Int. Cl. A61B 17/88

(52) U.S. Cl. CPC A61B 17/8875
USPC 606/104

(57) ABSTRACT

A tool for installing a bone screw in a bone. The tool has a guide wire having an inner end adapted to be seated at the bone at a site where the screw, which is tubular and can fit around and slide along the wire, is to be installed, a tubular tool body fittable over and around the wire and having a tip fittable with the bone screw when same is traversed by the wire to rotate and drive the bone screw, a stabilizer axially and rotationally fixable to the wire offset from the tool body so that the screw can be screwed axially into the bone at the site while the wire is prevented from moving axially by the stabilizer.

### FIELD OF THE INVENTION

The present invention relates to an installation tool for a bone screw. More particularly this invention concerns a surgical tool used to implant a bone screw in a vertebra or the like.

### BACKGROUND OF THE INVENTION

A typical installation tool for a bone screw, in particular for a pedicle screw of a spinal implant, has a head and a tubular body through which passes a guide wire. Tools of this type have long been used, among other things, for anchoring spinal implants and specifically with minimally invasive procedures.

In use of the tool, first a guide wire is inserted into the bone and the position thereof is monitored by an imaging system. Then, a bone screw and an installation tool, both of which are tubular for this purpose, are fitted one after the other on the guide wire in order to be guided to the installation site on the bone for the screw. In order to thereby facilitate the handling of the bone screw, it is generally releasably secured to the installation tool. This procedure allows an accurate and secure anchoring of the bone screw in the bone is facilitated thereby, since the bone screw and the installation tool always remain in contact with each other. Screwing the bone screw into the bone of the patient, however, always involves the risk of the guide wire being axially shifted during the screwing operation. The guide wire could thereby pierce the bone on the side lying opposite the screw and thereby possibly crack the bone or damage tissue.

For this reason it is usual in the case of conventional operating methods for the bone screw initially to be screwed in only by a few threads and the guide wire to then be removed from the bone before the bone screw is screwed home. However, this always results in the problem that while the bone screw is being screwed in, its position is no longer well defined, since the guide wire is no longer controlling its position, which can have a negative effect on the operation result.

### OBJECTS OF THE INVENTION

It is therefore an object of the present invention to provide an improved bone-screw installation tool.

Another object is the provision of such an improved an is improved bone-screw installation tool that overcomes the above-given disadvantages, in particular that allows the accurate positioning of the bone screw by leaving the guide wire in place without the danger of the guide wire being pushed further into or through the bone.

### SUMMARY OF THE INVENTION

A tool for installing a bone screw in a bone. The tool has according to the invention a guide wire having an inner end adapted to be seated at the bone at a site where the screw, which is tubular and can fit around and slide along the wire, is to be installed, a tubular tool body fittable over and around the wire and having a tip fittable with the bone screw when same is traversed by the wire to rotate and drive the bone screw, a stabilizer axially and rotationally fixable to the wire offset from the tool body so that the screw can be screwed axially into the bone at the site while the wire is prevented from moving axially by the stabilizer.

Thus with the installation tool of this invention the guide wire can now remain the patient's bone without moving while the bone screw is screwed in, since the axial position of the guide wire is fixed by the stabilizer, so that a piercing through the bone by the guide wire is no longer possible.

It has proven to be advantageous if the stabilizer has a passage to accommodate the guide wire. The wire is locked axially and angularly to the stabilizer so that deformation and thus a bending of the guide wire are avoided.

Furthermore, it has proven to be favorable if the stabilizer is formed by a body and a locking screw clamping the guide wire in the throughgoing bore or passage. The locking torque applied can be adjusted very exactly by use of a locking screw.

Furthermore, it has proven to be particularly favorable if the body has a threaded hole opening radially inwardly into the passage. An extremely simple and at the same time secure attachment method is provided by screwing in the locking screw, since the guide wire is clamped in the passage by the end of the locking screw. However, it is also within the scope of the invention for the locking screw to be embodied as a threaded cap that is screwed coaxially to the passage onto the body of the stabilizer, whereby a sealing ring supported between the body and the locking screw is deformed such that it clamps the guide wire.

It has furthermore proven to be advantageous if a threaded part of the stabilizer and a complementary thread is provided on the tool body. By the use of a threaded connection of this type, before the bone screw is screwed into the bone it is necessary only for the guide wire to be locked in the stabilizer and for the stabilizer to be held against rotation during the screwing. Thus when the bone screw is screwed into the bone of the patient, the axial position of the stabilizer changes relative to the tool body. Due to the coupling of the guide wire to the stabilizer on the one side and the coupling of the bone screw to the tool head on the other side, the axial position of the bone screw is naturally also changed relative to the guide wire while being screwing in. This ensures that the position of the guide wire always remains the same while the bone screw is screwed into the bone and piercing by the guide wire through the bone is avoided.

Furthermore, it has proven to be particularly favorable if the threaded part has approximately the same pitch and/or length as the screw thread of the bone screw. Due to this design of the pitch, while the bone screw is screwed into the bone, the position of the guide wire relative to the bone screw is changed such that the axial position of the guide wire in the bone does not change. Of course, it is also within the scope of the invention that the threaded part is embodied such that by screwing the bone screw into the bone a slight retraction of the guide wire is already given, whereby the guide wire after screwing in can be more easily removed from the bone without, however, losing the guiding effect on the bone screw.

Furthermore, it has proven to be advantageous if a frustoconical tip for the facilitated introduction of the guide wire is assigned to the stabilizer at its proximal end. Through the embodiment of a pointed tip of this type on the proximal end, that is the end toward the patient, guiding results that, during the placement of the installation tool on the guide wire, ensures that it can be easily inserted into the stabilizer and bending of the guide wire is avoided.

It has furthermore proven to be favorable if the distal end of the stabilizer has a grip part. This grip part makes it easier for the operator when screwing in the bone screw to ensure relative rotation between the tool body and the stabilizer.

It has proven to be particularly advantageous if the stabilizer is provided on the distal side of the tool, that is the tool body lies between the stabilizer and the screw. Due to the distal position, i.e. facing away from the patient, the stabilizer is easily accessible, which has a positive effective on the ease of handling the installation tool according to the invention.

It has furthermore proven to be favorable if the locking screw has a handle. The locking torque can be easily adjusted due to the embodiment of a handle on the self-locking screw. Furthermore, it also facilitates relative rotation of the tool body and the stabilizer when screwing in the bone screw.

It has furthermore proven to be advantageous if the handle is mounted perpendicular on the stabilizer. This has a positive effect on the ease of handling the installation tool according to the invention.

It has furthermore proven to be favorable if the tool body is embodied in a multipart manner, in particular, a two-part manner, and preferably can be divided by a detaching mechanism. Due to the two-part structure, after the bone screw has been screwed into the patient, the tool body can thereby be divided, whereby the distal part, which is assigned to the stabilizer, can be removed together with the guide wire. The proximal part of the tool body can now initially remain in the patient's body together with the tool screw in order to be useful, for example, in the assembly of a rod that connects several bone screws to one another.

### BRIEF DESCRIPTION OF THE DRAWING

The above and other objects, features, and advantages will become more readily apparent from the following description, reference being made to the accompanying drawing in which:

FIG. 1 is an exploded view of an installation tool according to the invention with a guide wire and a bone screw;

FIG. 2 is a side view of the installation tool with placed bone screw and the guide wire already set in a bone;

FIG. 3 is a view like FIG. 2, but with the installation tool with the bone screw screwed into the bone;

FIG. 4 is a large-scale detail view of a stabilizer; and

FIG. 5 shows the installation tool with unscrewed stabilizer and with an additional screw safety sleeve.

### DETAILED DESCRIPTION

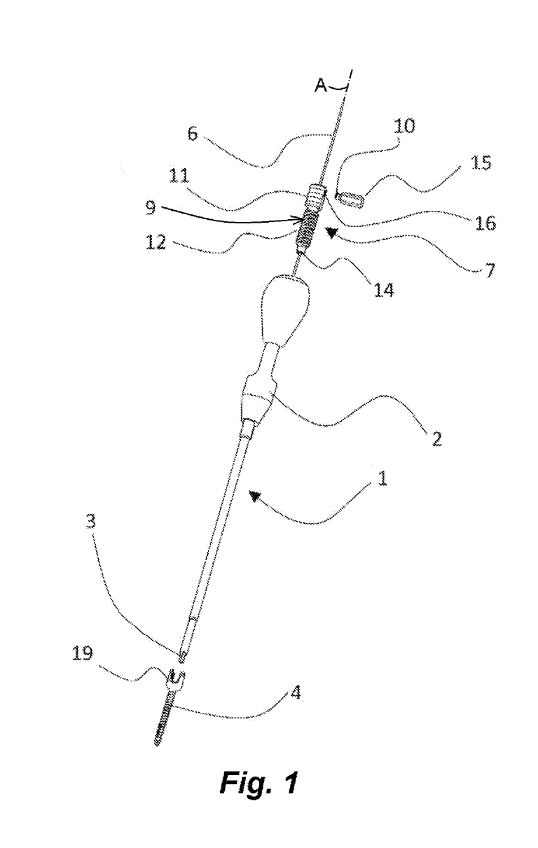

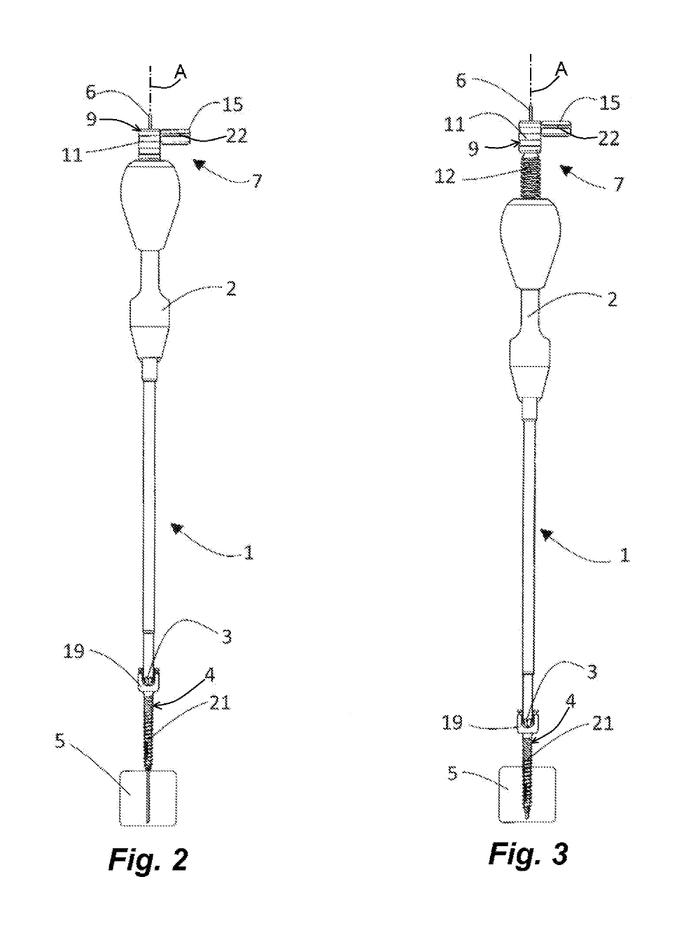

As seen in FIGS. 1 and 2, an installation tool 1 has a tubular tool body 2 and a tip 3 for fitting to and screwing a tubular bone screw 4 into a bone 5, all centered on an axis A. A stiff but flexible guide wire 6 is screwed into and seated in the bone 5 before the bone screw 4 touches the bone 5, and is thereafter used to guide the bone screw 4. For axial positioning of the guide wire 6 relative to the bone screw 4, a stabilizer 7 forming part of the installation tool 1 according to the invention has an axially throughgoing passage 8 to accommodate the guide wire 6.

In the illustrated embodiment, the stabilizer 7 has two parts: a tubular body 9 centered on the axis A and a locking screw 10 that engages in a threaded hole 16 extending radially in the body 9 and opening into the passage. The body 9 of the stabilizer 7 furthermore has two parts, namely on the distal end away from the patient an outer grip part 11 and, on the proximal end, a threaded and also tubular inner part 12 coaxial with the outer part 11. This part 11 is threaded into an internal screwthread 13 formed in the outer end of the passage 8 of the tool body 2. The screwthread-pitch and length of the threaded part 12 thereby corresponds essentially to the pitch and the length of a screw thread 21 of the bone screw 4.

For easy insertion of the guide wire 6 and stabilizer 7 into the tool body 2, the stabilizer body 9 has a frustoconically pointed proximal or inner end 14 facing toward the patient. In the embodiment shown, the locking screw 10 has a handle 15 formed with grip formations or wings handle 22 to facilitate turning the locking screw 10 into the threaded hole 16. Handling of the locking screw 10 is thus improved and furthermore holding of the stabilizer 7 while the bone screw 4 is screwed in is facilitated. Rotation of the stabilizer 7 relative to the tool body 2 can be easily prevented by gripping the stabilizer 7 with the handle 15 of the locking screw 10.

FIG. 2 shows the installation tool 1 according to the invention before insertion of the bone screw 4 into the bone 5. In the position shown in FIG. 2, the guide wire 6 was first inserted into the bone 5 and the position thereof verified by imaging. Then the tubular bone screw 4 and the also tubular installation tool 1 are slipped onto the guide wire 6 and the tool tip 3 is fitted to the bone screw 4. The guide wire 6 is then fixed in the body 9 of the stabilizer 7 by screwing in the locking screw 10, so that the axial position of the guide wire 6 relative to the bone screw 4 is fixed.

At this time the tool body 2 is gripped by the surgeon in one hand and the stabilizer 7 in the other, and the tool body 2 is rotated while the stabilizer is not. This causes the tool body to rotate the screw 4 and screw it into the bone 5, while simultaneously the body 2 screws itself off the threaded inner end 12 of the stabilizer as clearly shown in FIG. 3. Thus the position of the guide wire 6 is fixed in the bone 5 and does not change while the bone screw 4 is screwed in, since while the bone screw 4 is screwed in, the stabilizer 7 is simultaneously screwed out of the tool body 2, moving axially. When the screw 4 is in the desired end position, the entire assembly of tool body 2, stabilizer 7, and wire 6 are pulled or screwed backward out of the bone 5, leaving the screw 4 in position.

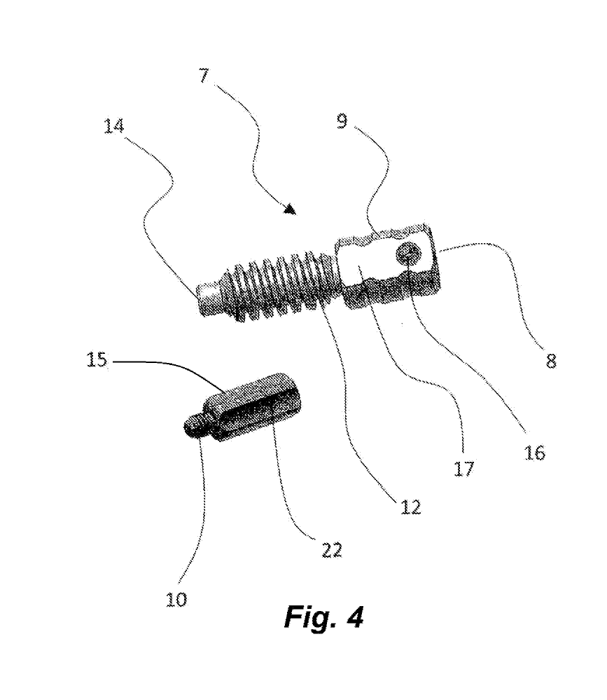

FIG. 4 shows a detailed view of the stabilizer 7 according to the invention. The division of the body 9 into two parts 11 and 12 can be clearly seen. The grip part 11 facilitates the holding of the stabilizer 7 and to this end has a structure that facilitates the manual gripping and holding to prevent its rotation when the tool body 2 is rotated about the axis A relative to the wire 6 to drive in the screw 4. Furthermore, the threaded hole 16 of the grip part 11 that opens into the passage in the stabilizer accommodating the guide wire 6 allows the locking screw 10 to clamp the guide wire 6 in the passage 8 and lock it rotationally and axially to the stabilizer 9. In order to facilitate insertion of the locking screw 10, the body 9 in the region of the grip part 11 has a flat 17 at which the bore 16 opens perpendicularly. Furthermore, at the proximal end of the stabilizer 7, the point tip 14 facilitates introduction of the guide wire 6 into the passage 8 of the stabilizer 7.

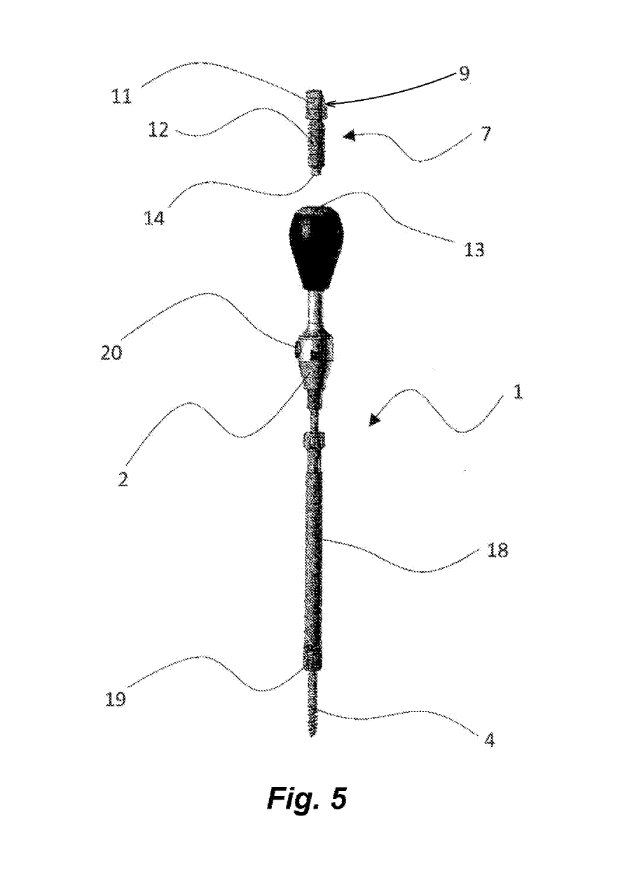

FIG. 5 shows a further embodiment of the installation tool 1 according to the invention. The tool body 2 is here embodied formed of two parts: a proximal or inner screw safety sleeve 18 that is supported in an axially displaceable manner on the proximal part of the tool body 2 and can be fitted with a screw head 19 of the bone screw 4. A solid unit can thus be produced between the tool body 2 and the bone screw 4, whereby both can be pushed onto the guide wire 5 simultaneously. This is used particularly in the case of polyaxial screws in order to be able to fix the position of the screw head 19. After the bone screw 4 has been screwed into the bone 5 of the patient, the tool body 2 can be separated by a detaching mechanism 20, whereby the guide wire 6 can be easily removed from the bone 2 and together with the stabilizer 7 and the distal part of the tool body 2 can be removed from the operating field. Now the screw safety sleeve 18 can be detached as required and the proximal part of the tool body 2 can be removed.

We claim:

1. A tool for installing a bone screw in a bone, the tool comprising:
a guide wire having an inner end adapted to be seated at the bone at a site where the screw, which is tubular and can fit around and slide along the wire, is to be installed;
a tubular tool body fittable over and around the wire and having a tip fittable with the bone screw when same is traversed by the wire to rotate and drive the bone screw;
a stabilizer axially and rotationally fixable to the wire offset from the tool body, whereby the screw can be screwed axially into the bone at the site while the wire is prevented from moving axially by the stabilizer.

2. The installation tool defined in claim 1, wherein the stabilizer has a stabilizer body formed with a throughgoing passage adapted to be traversed by the wire.

3. The installation tool defined in claim 2, wherein the stabilizer is provided with a locking screw for clamping the wire in the passage.

4. The installation tool defined in claim 3, wherein the stabilizer body is formed with a radial bore open radially outwardly, opening radially inward into the passage, and internally threaded to receive the screw.

5. The installation tool defined in claim 3, wherein the locking screw has an outer end formed with grip formations.

6. The installation tool defined in claim 3, wherein the locking screw and radial bore extend perpendicular to the axis.

7. The installation tool defined in claim 2, wherein the stabilizer body is formed with an axially extending and threaded part, the tool body having a complementarily threaded region receiving the threaded part of the stabilizer body such that relative rotation of the bodies about the axis axially displaces them relative to each other.

8. The installation tool defined in claim 7, wherein the bone screw and the screwthreads of the threaded part of the stabilizer body and of the tool body all have generally the same pitch such that as the screw is screwed axially inward a predetermined distance, the bodies move apart or together by a corresponding distance.

9. The installation tool defined in claim 2, wherein the tool body lies between the stabilizer and the screw.

10. The installation tool defined in claim 2, wherein the stabilizer body has a axially forwardly pointed inner end.

11. The installation tool defined in claim 2, wherein an outer end of the stabilizer body is formed as a hand grip.

12. The installation tool defined in claim 1, wherein the tool body has inner and outer parts joined by a detachable mount.

13. A tool for installing a tubular bone screw in a bone, the tool comprising:
a guide wire having an inner end adapted to be seated at the bone at a site where the screw and adapted to fit through the screw;
a tubular tool body fittable over and around the wire and having a tip fittable with the bone screw when same is traversed by the wire to rotate and drive the bone screw;
a tubular stabilizer traversed by the wire;
interengaging axially extending and complementary screwthread formations on the stabilizer and on the tool body that are screwed together such that relative rotation of the stabilizer and the tool body relatively axially displaces the stabilizer and tool body; and
means for axially and rotationally but releasably fixing the rod in the stabilizer, whereby the screw can be screwed axially into the bone at the site by relatively rotating the stabilizer and the tool body while the wire is prevented from moving axially by the relative axial movement of the stabilizer and tool body caused by the screwthread formations.
